(19) United States
(12) Patent Application Publication
(10) Pub. No.: US 2012/0271313 A1
(43) Pub. Date: Oct. 25, 2012

(54) LONG SCRAPER TOOL FOR DISCECTOMY

(75) Inventor: Craig E. Lauchner, Mountain View, CA (US)

(73) Assignee: Warsaw Orthopedic, Inc., Warsaw, IN (US)

(21) Appl. No.: 13/091,420

(22) Filed: Apr. 21, 2011

(51) Int. Cl. A61B 17/16

(52) U.S. Cl. 606/84

(57) ABSTRACT

A scraping instrument is disclosed that includes a handle assembly having a trigger. The scraping instrument also includes an elongate shaft having an inner lumen extending therethrough along a longitudinal axis between proximal and distal ends thereof. The proximal end of the elongate shaft is connected with the handle assembly. A control rod extends through the inner lumen of the elongate shaft hand has a first end connected with the trigger and a second end extending outside the distal end of the elongate shaft. The second end of the control rod includes a connection pin. A yoke is connected with the distal end of the elongate shaft. A curette having a scraping member and a connection member is pivotally connected with the yoke. Upon compression of the trigger the control rod is forced to move along the longitudinal axis thereby causing the curette to rotate along a latitudinal axis.

### BACKGROUND

The present invention relates generally to spinal fusion techniques, and more particularly to a long scraper tool for use in connection with performing a discectomy between vertebral bodies.

Spinal fusion is a surgical technique used to join two or more vertebrae. During this surgical technique, disc material is removed from the vertebrae. Complete discectomy is difficult to achieve with traditional tools. In particular, the nucleus area lateral and near the cannula is difficult to remove. In addition, the triangular area posterior and medial in the nucleus is also difficult to remove. As such, a need exists for instruments that can remove disc material during a spinal fusion procedure.

### SUMMARY

According to one aspect, a scraping instrument is disclosed that is used in performing a discectomy. The scraping instrument includes a handle assembly that includes a trigger. The scraping instrument also includes an elongate shaft having an inner lumen extending therethrough along a longitudinal axis between proximal and distal ends thereof. The proximal end of the elongate shaft is connected with the handle assembly. A control rod extends through the inner lumen of the elongate shaft and has a first end connected with the trigger and a second end extending outside the distal end of the elongate shaft. The second end of the control rod includes a connection pin and the control rod is movably positioned in the inner lumen. A yoke is connected with the distal end of the elongate shaft having a first arm and a second arm. A curette is included that has a generally rectangular shaped scraping member and a circular shaped connection member. The connection member is pivotally connected with the yoke between a gap defined by the first and second arms. The connection member is also connected with the connection pin of the control rod. Upon compression of the trigger, the control rod is forced to move along the longitudinal axis thereby causing the curette to rotate axially along a latitudinal axis.

In one form, the elongate shaft is connected with the handle assembly by a mounting block connected to a first frame of the handle assembly. In particular, the mounting block is connected to the first frame of the handle assembly by one or more screws that are connected to the first frame. The first end of the control rod includes an aperture and the trigger includes a control rod housing having a slot. A control rod pin is positioned in the aperture of the control rod thereby securing the control rod in the control rod housing.

The trigger is pivotally mounted to a first frame of the handle assembly by a trigger pivot pin. The trigger pivot pin is positioned in a boss located in the first frame of the handle assembly. In one form, each arm of the yoke includes an aperture sized and configured to receive a curette pivot pin. The curette is pivotally mounted to the yoke about the curette pivot pin. In yet another form, the scraping member has a length greater than a width associated with the scraping member of the curette. The longer, single, articulating scraper oriented normal to the vertebral endplates, can access difficult zones, achieving a more complete discectomy and thus a more robust fusion.

According to yet another aspect, a scraping instrument is disclosed for use in performing a discectomy. The scraping instrument includes a handle assembly comprising a first handle frame connected with a second handle frame. The handle assembly further includes a trigger pivotally mounted to the first and second handle frames. An outer tube is included having an inner lumen extending therethrough along a longitudinal axis between proximal and distal ends thereof. The proximal end of the outer tube is connected with the handle assembly. A control rod extends through the inner lumen having a first end connected with the trigger and a second end extending outside the distal end of the outer tube. The second end of the control rod includes a connector having a pin extending perpendicular to the longitudinal axis.

A yoke is connected with the distal end of the outer tube, wherein the yoke has a first arm and a second arm defining a gap therebetween. The second end of the control rod is positioned in the gap. A curette is included that has a scraping member and a connection member. The connection member includes an aperture sized and configured to receive the pin of the connector of the control rod. The connection member is pivotally connected with the first and second arms of the yoke such that upon activation of the trigger the control rod causes the curette to pivot about the first and second arms.

The trigger is pivotally mounted to at least the first handle frame by a trigger pivot pin. In one form, the trigger pivot pin is positioned in a trigger pin boss located on the first handle frame. A spring is included having a first end connected with a spring tab located on the trigger and a second end connected with the first handle frame. The outer tube is connected with the first handle frame of the handle assembly by a mounting block connected with the first handle frame that is configured to secure the outer tube to the handle assembly.

In one representative form, the first end of the control rod includes an aperture sized and configured to receive a trigger pin. The trigger includes a trigger pin receptacle sized and configured to receive the trigger pin thereby securing the control rod to the trigger. In one form, the scraping member has a generally rectangular shape. In this form, the scraping member has a length greater than a width associated with the scraping member. The connection member of the curette is pivotally mounted to the first and second arms of the yoke by a pivot pin.

In yet another aspect, a scraping instrument is disclosed that is configured and adapted for use in performing a discectomy. The scraping instrument includes a handle assembly having a first handle frame connected to a second handle frame. The handle assembly further includes a trigger pivotally mounted to the first handle frame. An elongate shaft is included having an inner lumen extending therethrough along a longitudinal axis between proximal and distal ends thereof. The proximal end of the elongate shaft is connected with the handle assembly. A control rod is included that extends through the inner lumen having a first end connected with the trigger and a second end extending outside the distal end of the elongate shaft. The second end of the control rod includes a connector portion having a pin. A yoke is included having an end sized and configured to be connected with the distal end of the elongate shaft. In addition, the yoke includes a first arm and a second arm extending outwardly from the end defining a gap therebetween. The first arm includes a first aperture and the second arm includes a second aperture.

The scraping instrument further includes a curette having a scraping member and a connection member. The connection member includes a third aperture in alignment with the first and second apertures of the yoke. The curette is pivotally connected with the yoke by a pivot pin positioned in the first, second and third apertures. The connection member is further connected with the pin of the connector portion of the control rod. Upon activation of the trigger, the control rod moves axially in the elongate shaft thereby causing the connection member to rotate which in turn causes the curette to rotate along a latitudinal axis.

The trigger includes a spring extension member and a first end of a spring is connected with the spring extension member and a second end of the spring is connected to the first handle frame thereby biasing the trigger in an open position. The trigger includes a control rod receptacle sized and configured to receive a first end of the control rod. The elongate shaft is mounted to the handle assembly by a mounting block. In one form, the scraping member has a length greater than a width associated with the scraping member and has a generally rectangular shape.

Related features, aspects, embodiments, objects and advantages of the present invention will be apparent from the following description.

### BRIEF DESCRIPTION OF THE DRAWINGS

FIG. 1 is a perspective view of a scraping instrument.

FIG. 2 is another perspective view of the scraping instrument illustrated in FIG. 1.

FIG. 3 is a perspective view of an elongate tube and curette of the scraping instrument illustrated in FIG. 1.

FIG. 4 is a side view of a portion of the scraping instrument illustrated in FIG. 1.

FIG. 5 is a side view of a portion of the scraping instrument illustrated in FIG. 1.

FIG. 6 is a side view of a portion of the trigger assembly of the scraping instrument illustrated in FIG. 1.

FIG. 7 is a perspective side view of a portion of a control rod and curette of the scraping instrument illustrated in FIG. 1.

FIG. 8 is a top view of a portion of a control rod, curette, and yoke of the scraping instrument illustrated in FIG. 1.

FIG. 9 is a side view of a portion of a control rod, curette, and yoke of the scraping instrument illustrated in FIG. 1.

FIG. 10 is a side view of a portion of the scraping instrument illustrated in FIG. 1.

### DESCRIPTION OF THE ILLUSTRATED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended. Any such alterations and further modifications in the illustrated devices, and such further applications of the principles of the invention as illustrated herein are contemplated as would normally occur to one skilled in the art to which the invention relates.

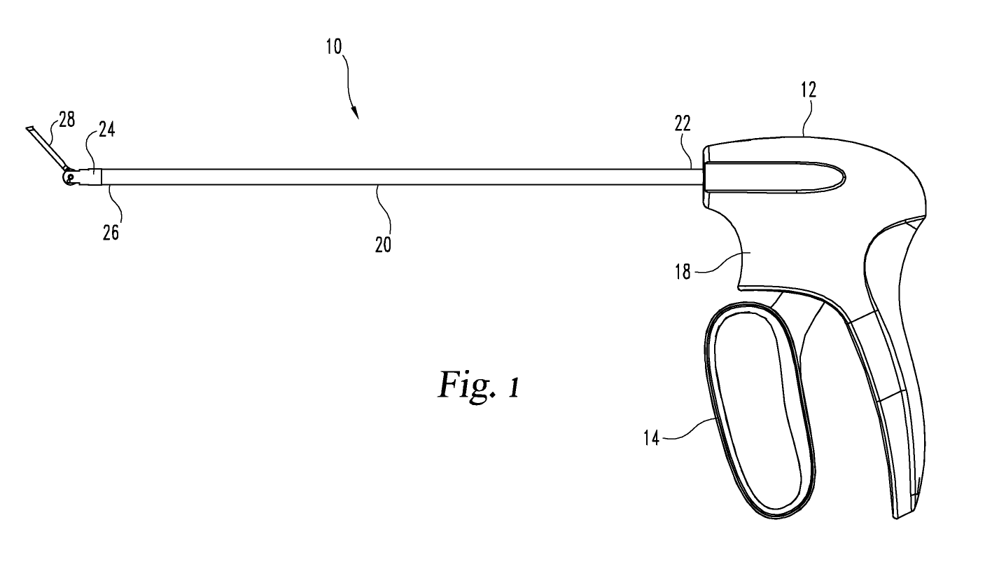

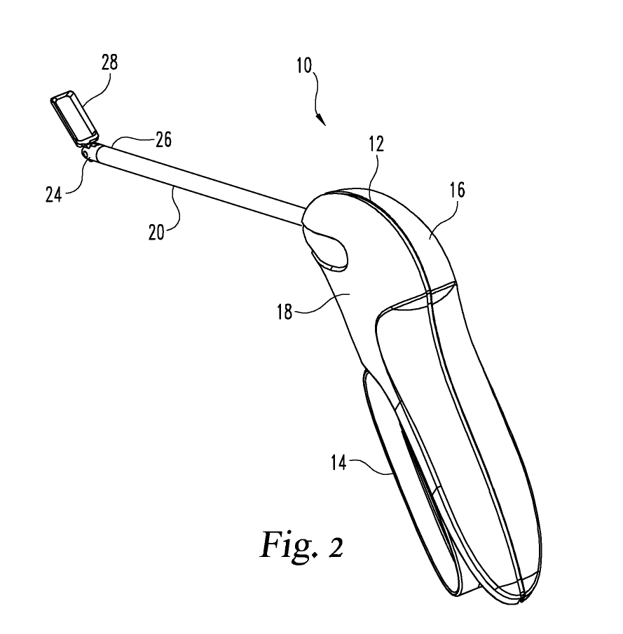

Referring to FIGS. 1 and 2, a perspective view of a scraping instrument 10 for use in connection with performing discectomy procedures is illustrated. The scraping instrument 10 includes a handle assembly 12 that includes a trigger 14. The handle assembly 12 is comprised of a first handle frame 16 that is connected with a second handle frame 18. An elongated shaft or outer tube 20 is connected with the handle assembly 12. A proximal end 22 of the elongated shaft 20 is connected with the handle assembly 12. A yoke 24 is connected with a distal end 26 of the elongated shaft 20. A curette 28 is connected with the yoke 24 that, as set forth in greater detail below, is operable to pivot about the yoke 24 when the trigger 14 is actuated.

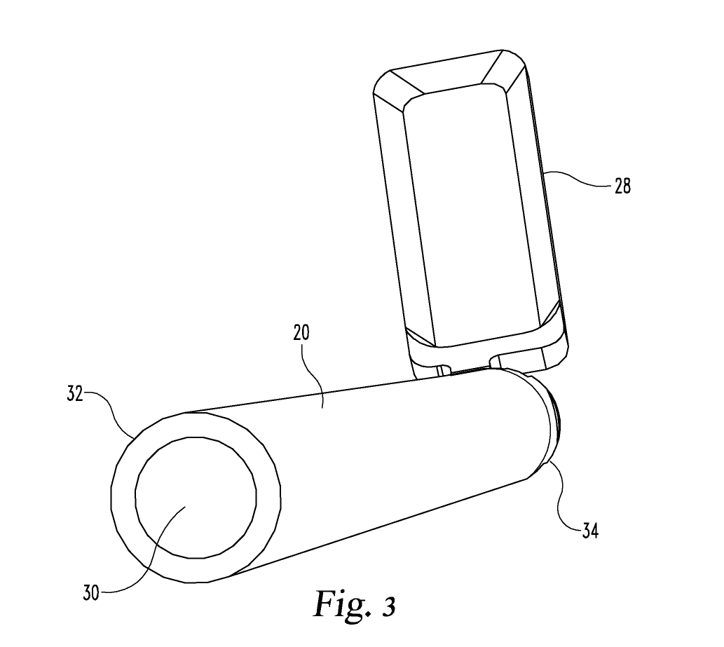

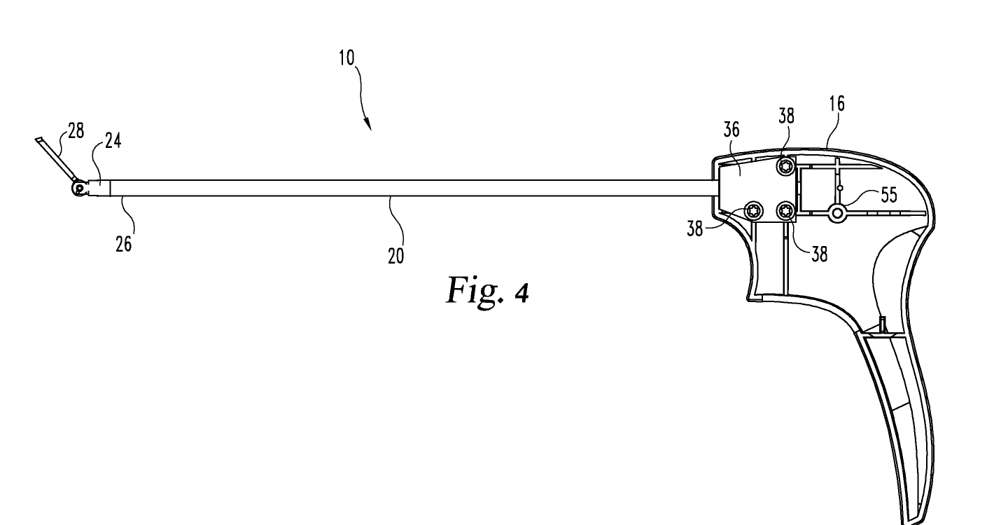

Referring to FIG. 3, the elongated shaft or outer tube 20 has an inner lumen 30 extending therethrough along a longitudinal axis between a proximal end 32 and a distal end 34. As illustrated in FIG. 4, the proximal end 32 of the elongated shaft 20 is connected with the handle assembly 12. In particular, a mounting block 36 is used to secure the elongated shaft 20 to the first handle frame 16. As depicted, the mounting block 36 is secured to the first handle frame 16 by a plurality of connection members or screws 38.

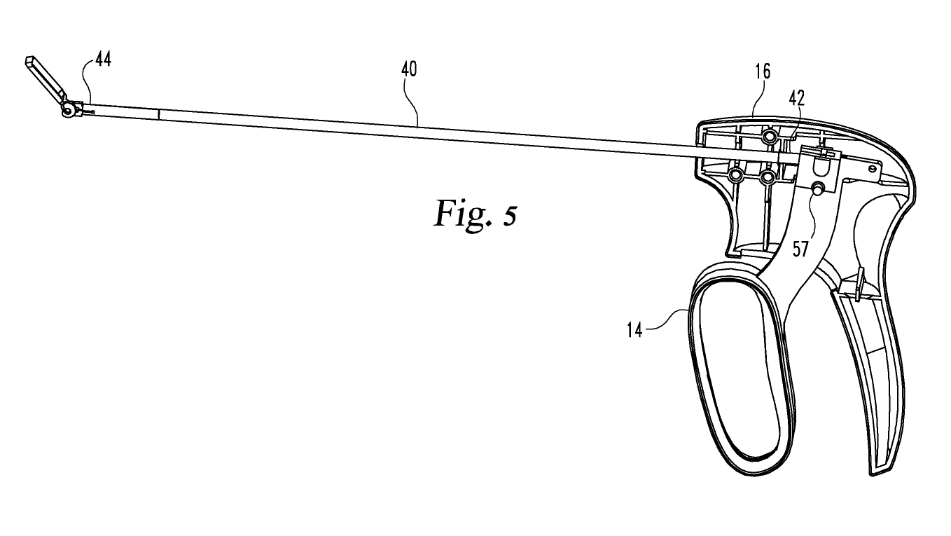

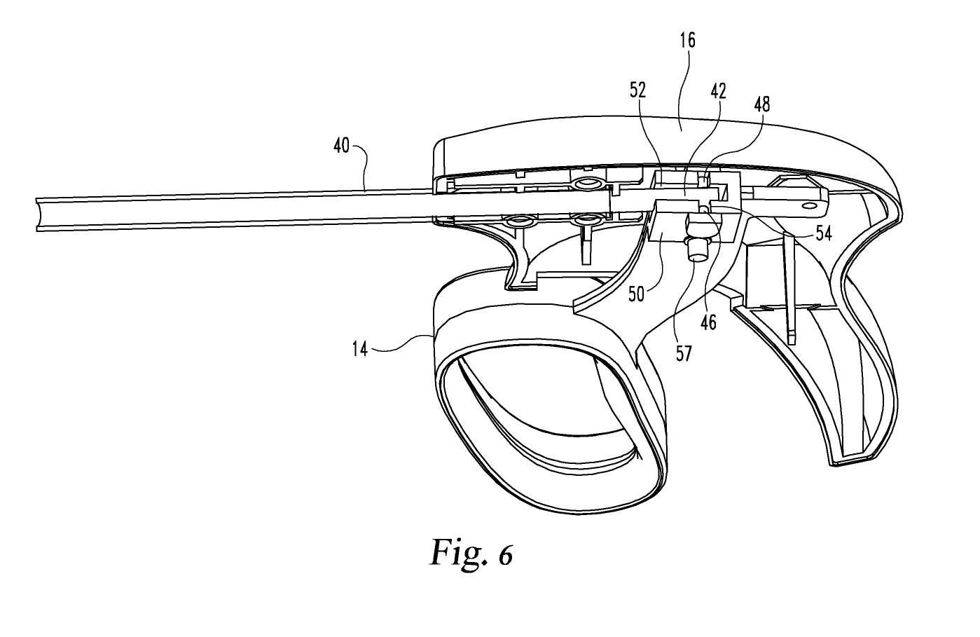

As illustrated in FIG. 5, a control rod 40 extends through the inner lumen 30 of the elongated shaft 20. The control rod 40 is sized such that it can move axially within the inner lumen 30 of the elongated shaft 20. The control rod 40 has a first end 42 connected with the trigger 14 and a second end 44 that extends outside the distal end 34 of the elongated shaft 20. As illustrated in FIG. 6, the first end 42 of the control rod 40 includes an aperture 46 that is sized and configured to receive a control rod pin 48. An upper portion 50 of the trigger 14 includes a control rod receptacle 52 that is sized and configure to receive a portion of the control rod 40 and the control rod pin 48. The receptacle 52 includes a control rod pin slot 54 that receives the control rod pin 48. The control rod pin 48, when positioned in the control rod pin slot 54, locks or secures the control rod 40 to the trigger 40.

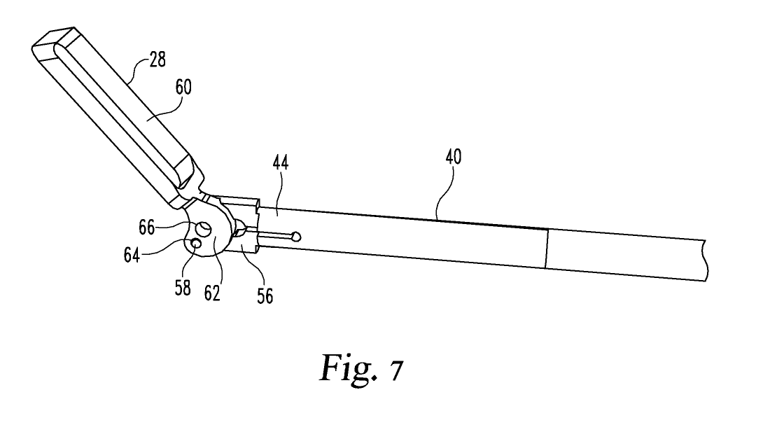

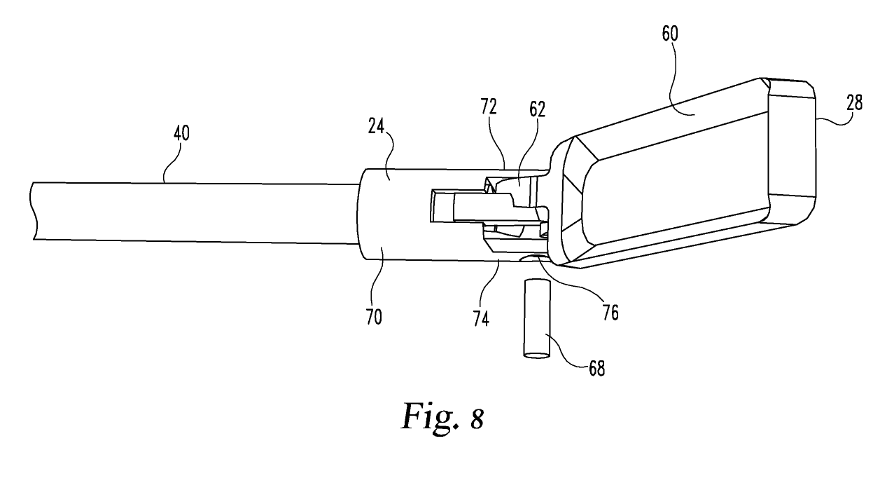

As illustrated in FIG. 7, the second end 44 of the control rod 40 includes a connection member 56. The connection member 56 includes a connection pin 58 that extends perpendicular to the longitudinal axis of the control rod 40. As illustrated, the curette 28 includes a scraping portion or member 60 and a curette connection member 62. A lower portion of the curette connection member 62 includes a first aperture 64 through which the connection pin 58 of the control rod 40 is positioned. As set forth in greater detail below, a central portion of the curette connection member 62 includes a second aperture 66 through which a pivot pin 68 (see FIG. 8) is positioned.

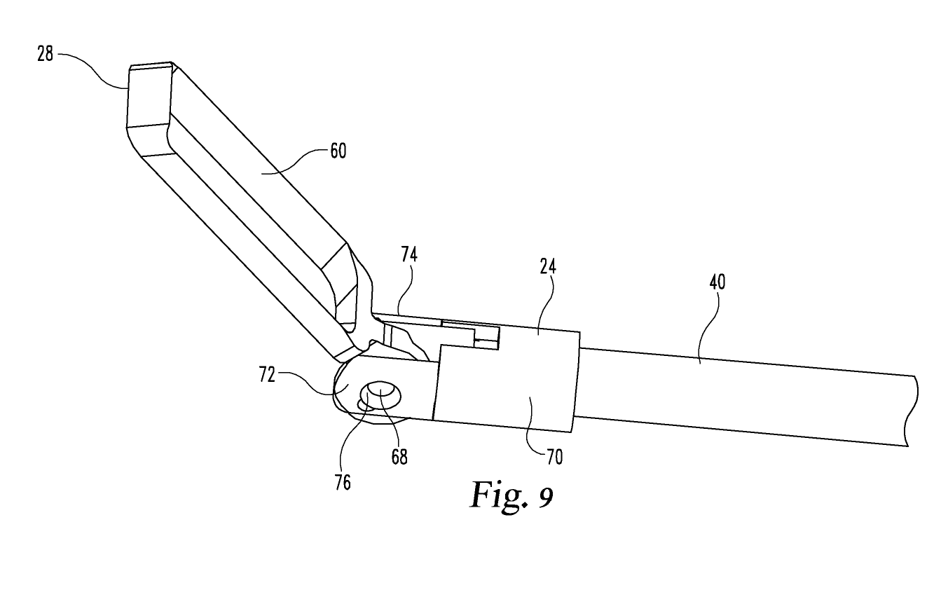

Referring to FIGS. 8 and 9, the yoke 24 includes a main body portion 70 that has a first arm 72 and a second arm 74 extending therefrom. As illustrated, the first arm 72 and second arm 74 define a gap between one another through which the second end 44 of the control rod 40 is movably inserted. Further, the first and second arms 72, 74 include a centrally located aperture 76 through which the yoke pivot pin 68 is inserted. The yoke pivot pin 68 is also inserted through the second aperture 66 of the curette connection member 62 thereby pivotally securing the curette 28 to the yoke 24. As the trigger 14 is actuated by a user, the control rod 40 moves forward along the longitudinal axis and the connection pin 58 of the control rod 40 forces the curette connection member 62 to pivot along the yoke pivot pin 68 thereby causing the curette 28 to move or pivot along a latitudinal axis.

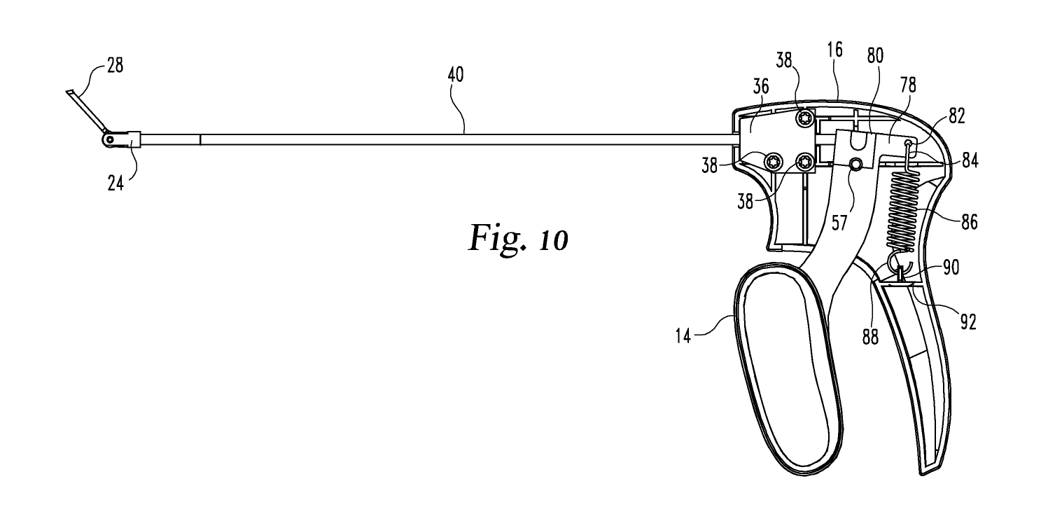

Referring to FIG. 10, the trigger 14 includes a spring tab 78 that protrudes outwardly from an upper end 80 of the trigger 14. The spring tab 78 includes an aperture 82 through which an upper end 84 of a coil spring 86 is connected. A lower end 88 of the coil spring 86 is connected with a frame spring tab 90 that is positioned in a central portion 92 of the first handle frame 16. The coil spring 86 biases the trigger 14 in an un-actuated state thereby causing the curette 28 to be oriented parallel to the longitudinal axis. As previously set forth, as the trigger 14 is actuated by the user, the curette 28 pivots upwardly about the yoke 24. As illustrated in FIG. 10, the curette 28 moves upwardly or horizontally along a latitudinal axis when the trigger 14 is actuated by the user.

Referring back to FIGS. 4 and 5, the trigger 14 includes a trigger pivot pin 57 that is positioned in a trigger boss 55. In one form, the trigger boss 55 is located in each of the first and second handle frames 16, 18 and the trigger pivot pin 57 is located in each respective trigger boss 55. The trigger pivot pin 57 allows the trigger 14 to pivot when actuated by the user. As previously set forth, the trigger 14 is utilized to actuate the control rod 40 which in turn causes the curette 28 to pivot about the yoke 24. As best illustrated in FIG. 7, in one form the scraping member 60 of the curette 28 has a generally rectangular shape or configuration. The scraping member 60 has a length greater than a width associated with the scraping member 60. This allows the scraping member 60 to be turned normal to the endplates and pulled proximally to remove otherwise inaccessible nucleus material. The scraping instrument 10 allows for a more complete discectomy, and thus a better final fusion between vertebral bodies.

Although various embodiments have been described as having particular features and/or combinations of components, other embodiments are possible having a combination of any features and/or components from any of embodiments as discussed above. As used in this specification, the singular forms “a,” “an” and “the” include plural referents unless the context clearly dictates otherwise. Thus, for example, the term “a member” is intended to mean a single member or a combination of members, “a material” is intended to mean one or more materials, or a combination thereof. Furthermore, the terms “proximal” and “distal” refer to the direction closer to and away from, respectively, an operator (e.g., surgeon, physician, nurse, technician, etc.) who would insert the medical implant and/or instruments into the patient. For example, the portion of a medical instrument first inserted inside the patient's body would be the distal portion, while the opposite portion of the medical device (e.g., the portion of the medical device closest to the operator) would be the proximal portion.

While the invention has been illustrated and described in detail in the drawings and foregoing description, the same is to be considered as illustrative and not restrictive in character, it being understood that all changes and modifications that come within the spirit of the invention are desired to be protected.

What is claimed is:

1. A scraping instrument, comprising:
a handle assembly having a trigger;
an elongate shaft having an inner lumen extending therethrough along a longitudinal axis between proximal and distal ends thereof, wherein said proximal end of said elongate shaft is fixedly connected with said handle assembly;
a control rod extending through said inner lumen of said elongate shaft having a first end connected with said trigger and a second end extending outside said distal end of said elongate shaft, wherein said second end of said control rod includes a connection pin;
a yoke connected with said distal end of said elongate shaft having a first arm and a second arm;
a curette having a scraping member and a connection member, wherein said connection member is pivotally connected with said yoke between said first and second arms, wherein said connection member is connected with said connection pin of said control rod, wherein upon compression of said trigger said control rod is forced to move along said longitudinal axis thereby causing said curette to rotate along a latitudinal axis.

2. The scraping instrument of claim 1, wherein said elongate shaft is fixedly connected with said handle assembly by a mounting block connected to a first frame of said handle assembly.

3. The scraping instrument of claim 1, wherein said first end of said control rod includes an aperture and said trigger includes a control rod housing having a slot, wherein a control rod pin is positioned in said aperture thereby securing said control rod in said slot of said control rod housing.

4. The scraping instrument of claim 1, wherein said trigger is pivotally mounted to a first frame of said handle assembly by a trigger pivot pin.

5. The scraping instrument of claim 1, wherein each arm of said yoke includes an aperture sized and configured to receive a curette pivot pin, wherein said curette is pivotally mounted to said yoke about said curette pivot pin.

6. The scraping instrument of claim 1, wherein said scraping member has a length greater than a width associated with said curette.

7. A scraping instrument, comprising:
a handle assembly comprising a first handle frame connected with a second handle frame, wherein said handle assembly further includes a trigger pivotally mounted to said first handle frame;
an outer tube having an inner lumen extending therethrough along a longitudinal axis between proximal and distal ends thereof, wherein said proximal end of said outer tube is connected with said handle assembly;
a control rod extending through said inner lumen having a first end connected with said trigger and a second end extending outside said distal end of said outer tube, wherein said second end of said control rod includes a connector having a pin extending perpendicular to said longitudinal axis;
a yoke connected with said distal end of said outer tube, wherein said yoke has a first arm and a second arm defining a gap therebetween, wherein said second end of said control rod is positioned in said gap;
a curette having a scraping member and a connection member, wherein said connection member includes an aperture sized and configured to receive said pin of said connector of said control rod, wherein said connection member is pivotally connected with said first and second arms of said yoke such that upon activation of said trigger said control rod causes said curette to pivot about said first and second arms.

8. The scraping instrument of claim 7, wherein said trigger is pivotally mounted to said first handle frame by a trigger pivot pin.

9. The scraping instrument of claim 8, wherein said trigger pivot pin is positioned in a trigger pin boss located on said first handle frame.

10. The scraping instrument of claim 7, further comprising a spring having a first end connected with a spring tab located on said trigger and a second end connected with said first handle frame.

11. The scraping instrument of claim 7, wherein said outer tube is connected with said first handle frame of said handle assembly by a mounting block connected with said first handle frame that is configured to secure said outer tube to said handle assembly.

12. The scraping instrument of claim 7, wherein said first end of said control rod includes an aperture sized and configured to receive a trigger pin, wherein said trigger includes a trigger pin receptacle sized and configured to receive said trigger pin thereby securing said control rod to said trigger.

13. The scraping instrument of claim 7, wherein said scraping member has a generally rectangular shape.

14. The scraping instrument of claim 7, wherein said scraping member has a length greater than a width associated with said scraping member.

15. The scraping instrument of claim 7, wherein said connection member of said curette is pivotally mounted to said first and second arms of said yoke by a pivot pin.

16. A scraping instrument, comprising:
a handle assembly having a first handle frame connected to a second handle frame, wherein said handle assembly further includes a trigger pivotally mounted to said first handle frame;
an elongate shaft having an inner lumen extending therethrough along a longitudinal axis between proximal and distal ends thereof, wherein said proximal end of said elongate shaft is connected with said handle assembly;
a control rod extending through said inner lumen having a first end connected with said trigger and a second end extending outside said distal end of said elongate shaft, wherein said second end of said control rod includes a connector portion having a pin;
a yoke having an end sized and configured to be connected with said distal end of said elongate shaft, wherein said yoke includes a first arm and a second arm extending outwardly from said end defining a gap therebetween, wherein said first arm includes a first aperture and said second arm includes a second aperture; and
a curette having a scraping member and a connection member, wherein said connection member includes a third aperture in alignment with said first and second apertures of said yoke, wherein said curette is pivotally connected with said yoke by a pivot pin positioned in said first, second and third apertures, wherein said connection member is further connected with said pin of said connector portion of said control rod, wherein upon activation of said trigger said control rod moves axially in said elongate shaft thereby causing said connection member to rotate which in turn causes said curette to rotate along a latitudinal axis.

17. The scraping instrument of claim 16, wherein said trigger includes a spring extension member, wherein a first end of a spring is connected with said spring extension member and a second end of said spring is connected to said first handle frame thereby biasing said trigger in an open position.

18. The scraping instrument of claim 16, wherein said trigger includes a control rod receptacle sized and configured to receive said first end of said control rod.

19. The scraping instrument of claim 16, wherein said elongate shaft is mounted to said handle assembly by a mounting block.

20. The scraping instrument of claim 16, wherein said scraping member has a length greater than a width associated with said scraping member.
